(19) United States
(12) Patent Application Publication
(10) Pub. No.: US 2006/0040344 A1
(43) Pub. Date: Feb. 23, 2006

(54) Microbial process for the preparation of 7-keto dehydroepiandrosterone and related analogs

(76) Inventors: Michael Jon White, Portage, MI (US); Doris M. Beck, Kalamazoo, MI (US); Ivan Gale Gilbert, Kalamazoo, MI (US)

Correspondence Address: WARNER-LAMBERT COMPANY, 2800 PLYMOUTH RD, ANN ARBOR, MI, 48105, US

(21) Appl. No.: 10/540,370

(22) PCT Filed: Jun. 21, 2004
(86) PCT No.: PCT/IB04/02078

§ 371 Date: Jun. 23, 2005

Related U.S. Application Data
(60) Provisional application No. 60/483,987, filed on Jul. 1, 2003.

(51) Int. Cl. C12P 33/06

(52) U.S. Cl. 435/58

(57) ABSTRACT

The present invention relates to a two part microbial process for the preparation of 7-oxo-5-androstene steroids of Formula III.

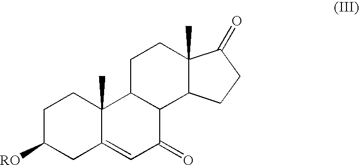

### BACKGROUND OF THE INVENTION

1. Field of the Invention

The present invention relates to a two part microbial process for the preparation of 7-oxo-5-androstene steroids of Formula III, 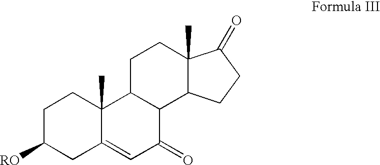
wherein:

R is H or —COR′;

R′ is alkyl of 1 to 5 carbons.

2. Background of the Invention

5-Androsten-3β-ol-7,17-dione (7-keto DHEA) is a commercially available steroid supplement that has been postulated to have beneficial effects on heart disease, immune function, aging, and general well-being.

U.S. Pat. No. 5,869,709 and Lardy, et al. (Steroids 63: 158-165, 1998) describe chemical processes for the synthesis of 7-keto DHEA and related analogues. There is no prior art describing a practical microbial method for the preparation of 7-keto DHEA.

Several methods exist detailing microbial 7α-hydroxylation of DHEA. Cotillon and co-workers (French Patent 2,771,105; J. Steroid Biochem. Molec. Biol. 62: 467-475, 1997; J. Steroid Biochem. Molec. Biol. 68; 229-237, 1999) use the filamentous fungus Fusarium monoliforme to effect 7α-hydroxylation of DHEA. While they detail high rates of conversion of DHEA to the desired product (greater than 80%), the substrate concentrations used are low. Kolek (J. Steroid Biochem. Molec. Biol. 71: 83-90, 1999) has studied hydroxylation of 5-ene steroids using Fusarium culmorum. It was observed that when the 5-ene steroid contained oxygen functions at C₃ and C₁₇, as with DHEA, the fungus hydroxylated entirely at the 7α-axial position. Again, the concentration of DHEA used in the study was low (20 mg per 80-mL culture, i.e., 0.25 g/L). Wilson, et al. (Steroids 64: 834-843, 1999) isolated Fusarium oxysporum var. cubense capable of 7α-hydroxylating 3β-hydroxy-Δ⁵-steroids. However, only a 68% product yield was achieved using a 0.8 g DHEA per liter of culture. Madyastha and Joseph (Appl. Microbiol. Biotechnol. 44: 339-343, 1995) used the filamentous fungus Mucor piriformis to transform DHEA. At a low substrate concentration (0.5 g/L), approximately 25% remained unconverted, and the rest was converted to several different oxidized and reduced products that included 7α-hydroxyl introduction.

7α-Hydroxysteroid dehydrogenase activity in bacteria such as Escherichia, Alcaligenes, Clostridium, Eubacterium, or Bacteroides, has been described (Schmidt, et al., J. Biol. Chem. 145: 229-236, 1942; Schmidt and Hughes, U.S. Pat. No. 2,360,447; MacDonald, et al., Analytical Chemistry 57: 127-136, 1974; MacDonald, et al., Applied Microbiology 30: 530-535, 1975; MacDonald, et al., Journal of Lipid Research 16: 244-246, 1975) for the oxidation of 7-hydroxy steroids to 7-oxo steroids. Recent research has focused on cloning and characterizing the bacterial 7α-hydroxysteroid dehydrogenase activity.

The microbial transformations described above are conducted using steroid substrate concentrations less than 0.5 grams per liter. Thus, there is a need for transformations using steroid substrate concentrations of 1 gram or greater per liter in order to provide a cost effective process for the preparation of 7-oxo-5-androstene steroids.

### DESCRIPTION OF THE INVENTION

### SUMMARY OF THE INVENTION

This invention relates to a two part process for preparing 7-oxo-5-androstene steroids. The first part comprises the steps of:

1) contacting a 5-androstene steroid of Formula I 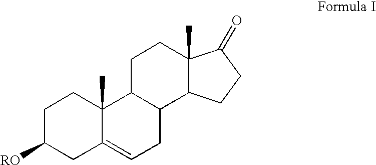
wherein:

R is H or —COR′;

R′ is alkyl of 1 to 5 carbons;
with a species of Mucor capable of performing the 7α-hydroxylation in a liquid culture at a steroid concentration of 1 gram per liter or greater to give a 7α-hydroxy-5-androstene steroid of Formula II;

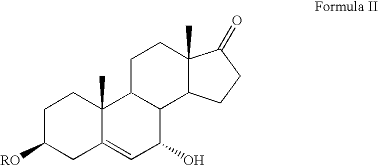

2) isolating the 7α-hydroxy-5-androstene steroid of Formula II from the liquid culture of step 1). The second part comprises the steps of:

3) contacting a 7-hydroxy-5-androstene steroid of Formula II in a liquid culture with any bacterium belonging to the genus Escherichia, Alcaligenes, Clostridium, Eubactenium, or Bacteroides, containing a 7α-hydroxysteroid dehydrogenase capable of oxidizing 7α-hydroxy-5-androstene steroids to a 7-oxo-5-androstene steroid of Formula III;

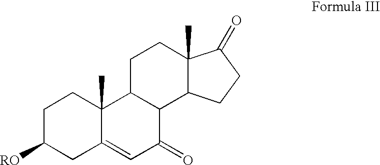

4) isolating the steroid of Formula m from the liquid culture.

### DETAILED DESCRIPTION OF THE INVENTION

### Description of the 7α-Hydroxylation Step

Any filamentous fungus of the genus Mucor capable of hydroxylating 5-androsten-3β-ol-17-one or related analogue of Formula I to produce 5-androsten-3β,7α-diol-17-one or related analogue of Formula II in high yield can be used in the invention process. The methods described in the examples may be used to determine the suitability of the filamentous fungus belonging to the genus Mucor. Preferably, Mucor rouxii is used. More preferably, Mucor rouxii ATCC 44260 (synonym NRRL 1894) is used.

The fungus of the genus Mucor that is contracted with the compound of Formula I may be utilized in the form of an actively growing culture, a whole-cell concentrate, or a cell-free extract. Preferably, the fungus is grown in submerged culture under aerobic conditions using any art-recognized procedure, and the transformation performed in situ. More preferably, the desired fungus is grown in submerged culture under aerobic conditions as set forth below and, more specifically, as set forth in EXAMPLE 1-4 using the ingredients specified, or other suitable carbon and nitrogen sources as are known to those skilled in the art. Non-limiting examples of suitable carbon sources include monosacharides, disacharaides, trisacharides and sugar alcohols such as glycerol and glucitol. Non-limiting examples of suitable organic nitrogen sources include casein, cornsteep liquor, meat extract, fish meal and soy protein hydrolysate. Non-limiting examples of suitable inorganic nitrogen sources include potassium nitrate, ammonium chloride, sodium nitrite and the like. Generally, a primary and secondary vegetative seed procedure is used in preparation for the fungal transformation of 5-androstene steroids of Formula I to 5-androstene-7-hydroxy steroids of Formula II. Alternatively, a primary vegetative seed can be used directly to inoculate bioconversion media.

Primary vegetative seed cultures may be incubated for a period of 24 to 96 hours (preferably 48 hours) at a temperature between 20° and 37° (preferably 28°), and an initial pH between 3.0 and 8.0. Secondary vegetative seed medium is inoculated with 0.006% to 0.1% (v/v) primary vegetative seed culture, but typically 0.012% (v/v), and incubated for a period of 36 to 72 hours (preferably 48-60 hours) at a temperature between 20° and 37° (preferably 28°). The pH of the secondary seed medium can be between 3.0 and 8.0, but preferably between 3.0 and 5.0. The bioconversion medium, which can be the same or similar to the secondary vegetative seed medium, is inoculated with 1% to 10% (v/v) secondary vegetative seed culture (preferably 3% to 5%). Bioconversion fermentation conditions can be the same as those used for cultivation of the secondary vegetative seed culture.

After an initial incubation period of zero to 72 hours (preferably 12 to 24 hours), 5-androstene steroids of Formula I, preferably micronized, is added to the bioconversion culture. Micronized 5-androstene steroids of Formula I can be added as a dry powder or an aqueous slurry, either as a single addition, a series of additions, or a continual feed. It is preferred to use the 5-androstene steroids of Formula I at a concentration greater than 1 g/L, more preferably greater than 5 g/L, even more preferably greater than 15 g/L. Bioconversion of 5-androstene steroids of Formula I to form 5-androstene-7-hydroxy steroids of Formula II is allowed to proceed for between about 2 and about 6 days, but typically about 4 days.

Once the conversion of 5-androstene steroids of Formula I, to 5-androstene-7-hydroxy steroids of Formula II is complete, 5-androstene-7-hydroxy steroids of Formula II can be isolated using any one of a number of art-recognized procedures. Preferably, 5-androstene-7-hydroxy steroids of Formula II are isolated using procedures as set forth below and, more specifically, as set forth in EXAMPLE 5 using the solvents specified, or other suitable solvents as are known to those skilled in the art. Filtered or centrifuged beer solids are extracted using an organic solvent, such as methanol, acetone, butyl acetate, or methylene chloride, and 5-androstene-7-hydroxy steroids of Formula II are isolated by crystallization. Non-limiting examples of crystallization solvents include water, methanol, acetone, butyl acetate, methylene chloride, or combinations thereof The preferred extraction solvent is methylene chloride, and the preferred crystallization solvent is n-butyl acetate.

### Description of the 7α-Hydroxysteroid Oxidation Step

Any bacterium belonging to the genus Escherichia, Alcaligenes, Clostridium, Eubacterium, or Bacteroides, containing a 7α-hydroxysteroid dehydrogenase capable of oxidizing 5-androstene-7-hydroxy steroids of Formula II, to produce 5-androsten-7-oxo steroids of Formula III, can be used in the invention process. The methods described in the examples may be used to determine the suitability of the bacterium. Preferably, bacteria from the genus Escherichia are used. More preferably, strains of Echerichia coli are used. Even more preferably, Eschericia coli ATCC 29532 is used.

The bacterium of the genus Escherichia, Alcaligenes, Clostridium, Eubacterium, or Bacteroides, may be utilized in the form of an actively growing culture, a whole-cell concentrate, or a cell-free extract. Preferably, the bacterium is grown in submerged culture under aerobic conditions using any art-recognized procedure, and the transformation performed in situ. More preferably, the desired bacterium is grown in submerged culture under aerobic conditions as set forth below and, more specifically, as set forth in EXAMPLE 6 using the ingredients specified, or other suitable carbon and nitrogen sources as are known to those skilled in the art. Generally, a primary vegetative seed procedure is used in preparation for the fungal transformation of 5-androstene-7-hydroxy steroids of Formula II to 5-androsten-7-oxo steroids of Formula III.

Primary vegetative seed medium is inoculated with a single isolated bacterial colony and the culture incubated for a period of 6 to 72 hours (preferably 24 to 36 hours) at a temperature between 25° and 40° (preferably 37°), and an initial pH between 5.0 and 8.0 (preferably neutral). The bioconversion medium, which can be the same or similar to the primary vegetative seed medium, is inoculated with 1% to 10% (v/v) primary vegetative seed culture (preferably 3% to 5°). Bioconversion fermentation conditions can be the same as those used for cultivation of the primary vegetative seed culture.

After an initial incubation period of zero to 36 hours (preferably 6 to 12 hours), 5-androsten-3β,7α-diol-17-one or related analogue of formula (II), preferably micronized, is added to the bioconversion culture. 5-Androstene-7-hydroxy steroids of Formula II can be added as a dry powder or an aqueous slurry, either as a single addition, a series of additions, or a continual feed. It is preferred to use the micronized 5-androstene-7-hydroxy steroids of Formula II at a concentration greater than 1 g/L, more preferably greater than 5 g/L, even more preferably greater than 9.5 g/L. Bioconversion of 5-androstene-7-hydroxy steroids of Formula II to form 5-androsten-7-oxo steroids of Formula III is allowed to proceed for between about 2 and about 7 days, but typically about 6 days.

Once the conversion of 5-androstene-7-hydroxy steroids of Formula II to 5-androsten-7-oxo steroids of Formula III is complete, the 5-androsten-7-oxo steroids of Formula III can be isolated using any one of a number of art-recognized procedures. Preferably, 5-androsten-7-oxo steroids of Formula III are isolated using procedures as set forth below and, more specifically, as set forth in EXAMPLE 7 using the solvents specified, or other suitable solvents as are known to those skilled in the art. Filtered or centrifuged beer solids are extracted using an organic solvent, such as methanol, acetone, butyl acetate, or methylene chloride, and 5-androsten-7-oxo steroids of Formula III are isolated by crystallization. Non-limiting examples of crystallization solvents include water, methanol, acetone, butyl acetate, methylene chloride, or combinations thereof. The preferred extraction solvent is methylene chloride and the preferred crystallization solvent is methanol.

### DEFINITIONS

The definitions and explanations below are for the terms as used throughout this entire document including both the specification and the claims.

All temperatures are in degrees Celsius.

r.p.m. refers to revolutions per minute.

TLC refers to thin-layer chromatography.

HPLC refers to high pressure liquid chromatography.

psig refers to pounds per square inch gage.

DO refers to dissolved oxygen.

RO refers to reverse osmosis.

SLM refers to standard liters per minute.

VVM refers to volume per minute.

OUR refers to oxygen uptake rate.

When solvent mixtures are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding descriptions, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures, both as to reactants and as to reaction conditions and techniques.

### Example 1

### Bioconversion of 5-androsten-3β-ol-17-one to 5-androsten-3β,7α-diol-17-one Using a Submerged Culture of Mucor rouxiii ATCC 44260 at a 10-L Fermentation Scale

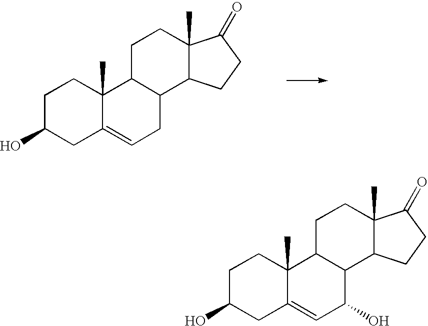

### (A) Primary-Seed Stage

Frozen vegetative cells of Mucor rouxii ATCC 44260 were thawed, transferred to potato-dextrose-agar plates (PDA), and incubated at 28° for 72 hours. Single mycelial-plugs (6-7 mm diam.) were used to moculate siliconized 500-mL stippled shake flasks containing 100 mL sterilized primary-seed medium. Primary-seed medium consisted of (per liter of RO water): dextrin, 50 g; soyflour, 35 g; cerelose, 5 g; coboalt chloride hexahydrate, 2 mg; silicone defoamer (SAG 471), 0.5 mL; pre-sterilization pH 7.0-7.1, adjusted with sodium hydroxide (2N). Mucor rouxii ATCC 44260 was incubated for 48 hours at 28°, using a controlled-environment incubator-shaker set at 275 rpm. (1″ orbital stroke).

### (B) Secondary-Seed Stage

Ten-liter secondary-seed fermentations were inoculated using 1.2 mL vegetative primary-seed culture (0.012% [v/v] inoculation rate). Secondary-seed medium contained (per liter of RO water): cerelose, 60 g; soyflour, 25 g; soybean oil, 5 mL; magnesium heptahydrate, 1 g; potassium dihydrogen phosphate, 0.74. g; silicone defoamer (SAG 471), 0.5 mL; pre-sterilization pH 4.95-5.00, adjusted with concentrated sulfuric acid. The fermentors, containing secondary-seed medium, were sterilized for 20 minutes at 121° using both jacket and injection steam. The agitation rate during sterilization was 200 r.p.m. Post-sterilization, the medium pH was adjusted to 5.0 using sterile sulfuric acid (5%). Mucor rouxii ATCC 44260 was incubated at 28° using the following initial parameters: agitation, 100 r.p.m.; back pressure=5 psig; airflow=2.5 SLM (0.25 VVM); low DO set-point, 50%; pH control, none. When the culture reached the low DO set-point, 50% DO was maintained using agitation control. Secondary-seed cultures were harvested at approximately 50 hours post-inoculation, when the OUR was between 8 and 10 mM/L/h.

### (C) Steroid Bioconversion

A ten-liter steroid-bioconversion fermentation was inoculated using 500 mL vegetative secondary-seed culture (5% [v/v] inoculation rate). Steroid-bioconversion medium contained (per liter of RO water): cerelose, 60 g; soyflour, 25 g; magnesium heptahydrate, 1 g; potassium dihydrogen phosphate, 0.74 g; silicone defoamer (SAG 471), 0.5 mL; pre-sterilization pH 3.95-4.00, adjusted with concentrated sulfuric acid. The fermentor, containing steroid-bioconversion medium, was sterilized for 20 minutes at 121° using both jacket and injection steam. The agitation rate during sterilization was 200 r.p.m. Post-sterilization, the medium pH was adjusted to 4.0 using sterile sulfuric acid (5%). Mucor rouxii ATCC 44260 was incubated at 28° using essentially the same initial parameters as those used for secondary-seed cultivation, with the exception that the initial agitation was 200 r.p.m. At approximately 19, 31, 43, and 55 hours post-inoculation, 50 g micronized 5-androsten-3β-ol-17-one slurried in a minimal volume of 0.2% octylphenoxy polyethoxy ethanol was added to the 10-L fermentation (total of 200 g 5-androsten-3β-ol-17-one).

The bioconversion culture was assayed on a daily basis for 5-androsten-3β,7α-diol-17-one using TLC. One milliliter of whole beer was diluted with 10 mL warm methanol. Cells were separated from the aqueous-methanol mixture by centrifugation (3,000×g for 10 minutes), and 5-10 μL applied to a TLC plate. The TLC plate was developed in methylene chloride:methanol:glacial acetic acid (95:5:1) and the product visualized by spraying the TLC with 50% sulfuric acid, followed by chainig in an oven. Product was compared to authentic standard, which turns blue on spraying with 50% sulfuric acid. Bioconversion of 5-androsten-3β-ol-17-one to 5-androsten-3β,7α-diol-17-one was complete in approximately 4 days.

### Example 2

### Bioconversion of 5-androsten-3β-ol-17-one to 5-androsten-3β,7α-diol-17-one Using a Submerged Culture of Mucor rouxiii ATCC 44260 at a 10-L Fermentation Scale

### (A) Primary-Seed Stage

Primary-seed cultures were prepared as described in EXAMPLE 1.

### (B) Secondary-Seed Stage

Ten-liter secondary-seed cultures were prepared as described in EXAMPLE 1.

### (C) Steroid Bioconversion

A ten-liter bioconversion was performed as described in EXAMPLE 1, except the medium pH was adjusted to 5.0. The bioconversion culture was assayed on a daily basis for 5-androsten-3β,7α-diol-17-one using TLC, as described in EXAMPLE 1. Bioconversion of 5-androsten-3β-ol-17-one to 5-androsten-3β,7α-diol-17-one was complete in approximately 4 days.

### Example 3

### Bioconversion of 5-androsten-3β-ol-17-one to 5-androsten-3β,7α-diol-17-one Using a Submerged Culture of Mucor rouxiii ATCC 44260 at a 10-L Fermentation Scale

### (A) Primary-Seed Stage

Primary-seed cultures were prepared as described in EXAMPLE 1.

### (B) Secondary-Seed Stage

Ten-liter secondary-seed cultures were prepared as described in EXAMPLE 1.

### (C) Steroid Bioconversion

A ten-liter bioconversion was performed as described in EXAMPLE 1, except the medium contained (per liter of RO water): cerelose, 60 g; soyflour, 35 g; dextrin, 50 g; silicone defoamer (SAG 471), 0.5 mL; pre-sterilization pH 3.95-4.00, adjusted with concentrated sulfuric acid. The bioconversion culture was assayed on a daily basis for 5-androsten-3β,7α-diol-17-one (II) using TLC, as described in EXAMPLE 1. Bioconversion of 5-androsten-3β-ol-17-one (1) to 5-androsten-3β,7α-diol-17-one was complete in approximately 4 days.

### Example 4

### Bioconversion of 5-androsten-3β-ol-17-one to 5-androsten-3β,7α-diol-17-one Using a Submerged Culture of Mucor rouxiii ATCC 44260 at a 10-L Fermentation Scale

### (A) Primary-Seed Stage

Primary-seed cultures were prepared as described in EXAMPLE 1.

### (B) Secondary-Seed Stage

Ten-liter secondary-seed cultures were prepared as described in EXAMPLE 1.

### (C) Steroid Bioconversion

A ten-liter bioconversion was performed as described in EXAMPLE 3, except the medium pH was adjusted to 5.0. The bioconversion culture was assayed on a daily basis for 5-androsten-3β,7α-diol-17-one using TLC, as described in EXAMPLE 1. Bioconversion of 5-androsten-3β-ol-17-one to 5-androsten-3β,7α-diol-17-one was complete in approximately 4 days.

### Example 5

### Isolation of 5-androsten-3β,7α-diol-17-one

The whole beer at harvest, from four 10-L fermentations (EXAMPLES 1-4), was centrifuged and the rich solids recovered by centrifugation. The rich solids were extracted using 40 liters of methylene chloride. The rich organic extract was separated from the solids by settling. The methylene chloride extract was polished and concentrated to approximately 2 L by distillation, and then 2 L of n-butyl acetate added. This mixture was concentrated to approximately 2 L and cooled to 4° to complete product crystallization. The crystals were recovered by filtration, washed with cold butyl acetate to remove color, and dried to give 496 g of crystalline 5-androsten-3β,7α-diol-17-one.

### Example 6

### Bioconversion of 5-androsten-3β,7α-diol-17-one to 5-androsten-3β-ol-7,17-dione Using a Submerged Culture of Escherichia coli ATCC 29532 at a 100-mL Fermentation Scale

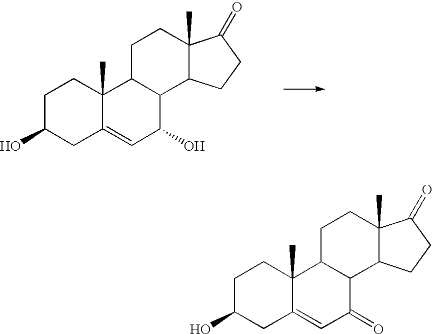

### (A) Primary-Seed Stage

Frozen vegetative cells of Escherichia coli ATCC 29532 were thawed, transferred to a Brain Heart Infusion agar plate, and incubated at 37° for 24 hours. Single bacterial colonies were used to inoculate 500-mL shake flasks containing 100 mL sterilized primary-seed medium. Primary-seed medium consisted of (per liter of RO water): hydrolyzed soy protein, 12 g; autolysed yeast extract, 24 g; glycerol, 5 mL; potassium phosphate monobasic (crystals), 2.31 g; potassium phosphate dibasic (anhydrous), 12.54 g; pH neutral (no adjustment). Escherichia coli ATCC 29532 was incubated for approximately 24 hours at 37°, using a controlled-environment incubator-shaker set at 270 rpm. (2″ orbital stroke).

### (B) Steroid Bioconversion

One hundred milliliter sterilized steroid-bioconversion medium was inoculated with 3 mL vegetative primary-seed culture (3% [v/v] inoculation rate). Steroid-bioconversion medium was the same as the primary-seed medium. Escherichia coli ATCC 29532 cultures were incubated at 37°, using a controlled-environment incubator-shaker set at 270 rpm. (2″ orbital stroke). At approximately 8 hours post-inoculation, 1 g micronized 5-androsten-3β,7α-diol-17-one slurried in a minimal volume of 0.2% polyoxyethylenesorbitan monooleate was added to the 100-niL fermentation.

Bioconversion cultures were assayed on a daily basis for 5-androsten-3β-ol-7,17-dione using TLC. One milliliter of whole beer was extracted with 1 mL warm methanol. Cells were separated from the aqueous-methanol mixture by centrifugation (3,000×g for 10 minutes), and 2 μL applied to a TLC plate. The TLC plate was developed in cyclohexane:ethylacetate:methanol:glacial acetic acid (90:60:30:1) and the product visualized first by UV₂₅₄, then by spraying with 50% sulfuric acid and charring in an oven. Product was compared to authentic standard, which absorbs UV light at 254 nm and turns yellow on charring with sulfuric acid. Bioconversion of 5-androsten-3β,7α-diol-17-one to 5-androsten-3β-ol-7,17-dione was complete in approximately 6 days.

### Example 7

### Isolation of 5-androsten-3β-ol-7,17-dione

The whole beer at harvest, from forty 100-mL fermentations (EXAMPLE 6), was centrifuged and the rich solids recovered by centrifugation. The rich solids were extracted using 3 L of methylene chloride. The rich organic extract was separated from the solids by centrifugation. The methylene chloride extract was polished and concentrated to approximately 250 mL by distillation, and then 500 mL methanol added. This mixture was concentrated to approximately 250 mL and cooled to −10 to complete product crystallization. The crystals were recovered by filtration, washed with cold methanol to remove color, and dried to give 29 g of crystalline 5-androsten-3β-ol-7,17-dione.

1. A process for preparing 7-hydroxy-5-androstene steroids comprising the steps of:
1) contacting a 5-androstene steroid of Formula I 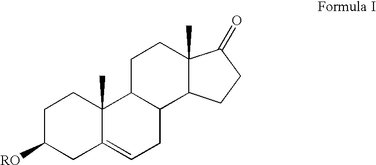
wherein:
R is H or —COR′;
R′ is alkyl of 1 to 5 carbons;
with a species of Mucor capable of performing a microbial transformation in a liquid culture at a steroid concentration of 1 gram per liter or greater to give a 7α-hydroxy-5-androstene steroid of Formula II; 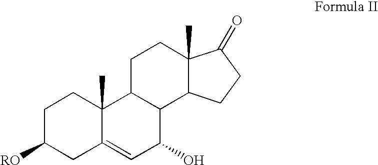
2) isolating the 7α-hydroxy-5-androstene steroid of Formula II from the liquid culture of step 1).

2. A process according to claim 1 further comprising the steps of:
3) contacting a 7-hydroxy-5-androstene steroid of Formula II in a liquid culture with any bacterium belonging to the genus Escherichia, Alcaligenes, Clostridium, Eubacterium, or Bacteroides, containing a 7α-hydroxysteroid dehydrogenase capable of oxidizing 7α-hydroxy-5-androstene steroids to a 7-oxo-5-androstene steroid of Formula III; 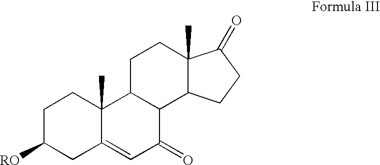
4) isolating the steroid of Formula III from the liquid culture of step 3.

3. A process for preparing 7-oxo-5-androstene steroids comprising the steps of:
1) contacting a 5-androstene steroid of Formula I 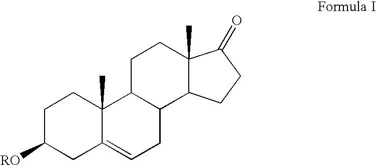
wherein:
R is H or —COR′;
R′ is alkyl of 1 to 5 carbons;
with a species of Mucor capable of performing a microbial transformation in a liquid culture to give a 7α-hydroxy-5-androstene steroid of Formula II; 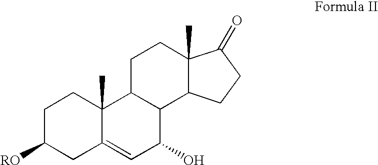
2) isolating the 7α-hydroxy-5-androstene steroid of Formula II from the liquid culture of step 1);
3) contacting a 7-hydroxy-5-androstene steroid of Formula II in a liquid culture with an Escherichia species capable of oxidizing 7α-hydroxy-5-androstene steroids to a 7-oxo-5-androstene steroid of Formula III; 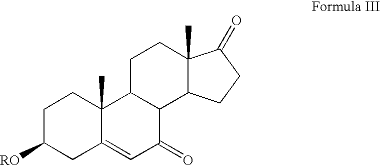
4) isolating the steroid of Formula III from the liquid culture of step 3.

4. A process according to claim 1 wherein the Mucor species is Mucor rouxii.

5. A process according to claim 1 wherein the Mucor species is Mucor rouxii ATCC 4260.

6. A process according to claim 2 wherein the Mucor species is Mucor rouxii.

7. A process according to claim 2 wherein the Mucor species is Mucor rouxii ATCC4260.
